(19) United States
(12) Patent Application Publication
(10) Pub. No.: US 2025/0226093 A1
(43) Pub. Date: Jul. 10, 2025

(54) PREDICTIVE MACHINE LEARNING MODELS FOR PREECLAMPSIA USING ARTIFICIAL NEURAL NETWORKS

(71) Applicant: Giovanni BELLESIA, San Carlos, CA (US)

(72) Inventors: Giovanni BELLESIA, San Jose, CA (US); Brittany PRIGMORE, Austin, TX (US); Ebad AHMED, San Jose, CA (US); Vivienne SOUTER, Seattle, WA (US); Asma KHALIL, London (GB)

(73) Assignee: Natera, Inc., San Carlos, CA (US)

(21) Appl. No.: 18/850,802

(22) PCT Filed: Mar. 28, 2023
(86) PCT No.: PCT/US23/16494

§ 371 Date: Sep. 25, 2024

Related U.S. Application Data
(60) Provisional application No. 63/324,469, filed on Mar. 28, 2022.
(60) Provisional application No. 63/400,380, filed on Aug. 23, 2022.

(51) Int. Cl. G16H 50/20; G16B 40/20; G16H 10/60

(52) U.S. Cl. CPC G16H 50/20; G16B 40/20; G16H 10/60

(57) ABSTRACT

Disclosed is an approach that may include generating and/or using a predictive machine learning classifier comprising one or more artificial neural networks. The classifier is configured to output a prediction related to developing preeclampsia (e.g., early onset preterm preeclampsia) during a current pregnancy of a patient based on health characteristics and one or more DNA metrics. The health characteristics and DNA metrics, such as total cell-free DNA (cfDNA) and fetal fraction (FF), may be obtained during a routine and non-invasive or minimally-invasive prenatal screening. The predictive machine learning classifier may be generated by applying deep learning techniques to data on subjects in a cohort. The data may comprise features corresponding to outcomes of prior pregnancies, health indicators, and one or more cfDNA measurements. First trimester risk assessment for preterm preeclampsia can identify patients most likely to benefit from preventative treatment protocols with a minimal or low level of intervention.

### CROSS-REFERENCE TO RELATED APPLICATIONS

This patent application claims priority to U.S. Provisional Patent Application 63/324,469, filed Mar. 28, 2022, and U.S. Provisional Patent Application 63/400,380, filed Aug. 23, 2022, the entirety of each of which is incorporated herein by reference.

### TECHNICAL FIELD

The present technology relates generally to using artificial intelligence to make predictions for a health outcome of a pregnant subject, and more specifically, to machine learning models for identifying patients at risk for developing preeclampsia during a current pregnancy using patient characteristics that, for example, are collected non-invasively or minimally-invasively during routine visits to healthcare providers.

### BACKGROUND

Preeclampsia is a major contributing factor to maternal mortality and morbidity. First trimester risk assessment for preterm preeclampsia (<37 weeks' gestation) can identify patients most likely to benefit from low dose aspirin. Low dose aspirin started between 12 and 16 weeks' gestation has been shown to decrease the risk of early onset preeclampsia in high risk patients. Accurate first trimester assessment of preeclampsia risk enables identification of patients most likely to benefit from initiation of aspirin at 12 to 16 weeks' gestation when there is evidence for its effectiveness. Risk assessment is also important for guiding appropriate pregnancy care pathways and surveillance.

Current United States Preventive Services Task Force (USPSTF) guidelines have limited clinical ability to identify patients at risk for preeclampsia and more accurate early pregnancy risk assessment has been identified as a priority. Previous approaches for predicting preeclampsia have used patient demographic and clinical characteristics, ultrasound measurements, and biomarkers. However, the more costly, difficult to obtain, invasive, and/or non-routine the health data that are needed to predict preterm preeclampsia, the more likely that at least some high-risk patients will not be identified because such health data are less commonly available.

### SUMMARY

Various potential embodiments of the disclosed approach relate to a method of applying, by a computing system, a predictive machine learning classifier to generate a prediction of a patient developing a preeclampsia condition, such as preterm preeclampsia or term preeclampsia, or other pregnancy outcomes during a current pregnancy. The predictive machine learning classifier may comprise one or more artificial neural networks. Generating the prediction may comprise obtaining patient data by at least one of (A) performing a non-invasive prenatal screening (NIPS) on the patient, and/or (B) receiving, by the computing system through a communications network, data from a health record system. The patient data may comprise (i) a set of health characteristics, and/or (ii) one or more cell-free DNA (cfDNA) metrics. As used herein, a cfDNA metric is any measured, derived, or computed value based on data generated from one or more cfDNA analyses. Examples of cfDNA metrics include, without limitation, total cfDNA, fetal fraction (FF), SNP data from cfDNA screening, etc. Generating the prediction may comprise feeding the patient data to the predictive machine learning classifier to obtain a prediction of the patient developing the preterm preeclampsia during the current pregnancy. The method may comprise providing, by the computing system, the prediction for care of the patient. Providing the prediction may comprise at least one of transmitting, by the computing system through the communications network, the prediction to at least one of the health record system and/or a computing device associated with a healthcare provider. The predictive machine learning classifier may have been trained by applying one or more machine learning techniques to data on subjects in a cohort of pregnant subjects. In various potential implementations, the one or more machine learning techniques can comprise, for example, deep learning, logistic regression, random forest, and/or gradient boosting. The data may comprise, for each pregnant subject, a plurality of: (A) a first feature set indicative of one or more outcomes of one or more prior pregnancies of the pregnant subject; (B) a second feature set comprising one or more health indicators for the pregnant subject; and/or (C) a third feature set based on one or more cfDNA measurements for the pregnant subject. As used herein, health indicators (used interchangeably with health characteristics or health data) comprise values (measured or derived) correlated with a health quality or wellness level, and include, without limitation, blood pressure, body mass index (BMI), weight, biomarkers (e.g., blood levels of various compounds), etc. As used herein, patient characteristics (used interchangeably with patient data) can include, or can be, health indicators and/or other information about a patient that, by itself, is not necessarily indicative of a health quality or wellness level. By non-limiting example, gender at birth could be a patient characteristic.

In various implementations, all of the patient data used to obtain the prediction may be collected non-invasively during one or more routine medical visits of the patient. In various implementations, the patient data used to obtain the prediction may exclude uterine artery Doppler and/or other ultrasound data. In various embodiments, the preeclampsia is preterm preeclampsia or early onset preterm preeclampsia. In various embodiments, the method may comprise generating, by the computing system, a training dataset based on the first, second, and/or third feature sets. In various embodiments, the method may comprise training, by the computing system, the predictive machine learning classifier using the data on the subjects in the cohort of pregnant subjects. In various embodiments, the predictive machine learning classifier may comprise, for example, any combination of one or more rectified linear units (reLu), sigmoid linear units (SiLU), softmax functions, etc. In various embodiments, the predictive machine learning classifier may comprise a hidden layer with a same dimension as an input layer. In various embodiments, the predictive machine learning classifier may comprise multiple hidden layers and may have varying dimensions. In various embodiments, the one or more deep learning techniques may comprise using one or more regularizers (e.g., an L2 regularizer).

Various potential embodiments of the disclosed approach relate to a method comprising generating a predictive machine learning classifier for preeclampsia in pregnant patients. The predictive machine learning classifier may comprise one or more artificial neural networks. Generating the prcdictivc machine learning classifier may comprisc: generating, by a computing system, a training dataset using data on subjects in a cohort of pregnant subjects. The training dataset may comprise, for each pregnant subject, a plurality of: (A) a first feature set indicative of one or more outcomes of one or more prior pregnancies of the subject; (B) a second feature set corresponding to one or more health indicators for the subject; and/or (C) a third feature set corresponding to one or more cfDNA metrics. The method may comprise applying, by the computing system, one or more deep learning techniques to the training dataset to generate the predictive machine learning classifier. The predictive machine learning classifier may be configured to receive, as inputs, patient health characteristics and one or more cfDNA metrics, and output predictions on developing preeclampsia. Patient health characteristics or other patient data may be obtained from or via, for example, an electronic medical record system. The method may comprise providing, by the computing system, the predictive machine learning classifier to generate patient-specific indicators for individual patients. Providing the predictive machine learning classifier may comprise at least one of (i) transmitting, by the computing system through a communications network, the predictive machine learning classifier to a second computing system, and/or (ii) storing, in a non-transient computer-readable storage medium, the predictive machine learning classifier accessible for subsequent use by a healthcare provider. The second computing system may be, for example, an electronic medical record system.

In various example implementations, the predictive machine learning classifier may comprise one or more hidden rectified linear units (reLu). In various implementations, the predictive machine learning classifier may comprise a hidden layer with a same dimension as an input layer. In various implementations, applying the one or more deep learning techniques may comprise using one or more regularizers. In various implementations, the one or more regularizers may comprise an L2 regularizer. In various implementations, the method may comprise using the predictive machine learning classifier to evaluate a patient during a current pregnancy by: performing a non-invasive prenatal screening (NIPS) on the patient; feeding data to the predictive machine learning classifier to obtain a prediction for development of the preeclampsia during the current pregnancy, the data comprising data from the NIPS; and/or using the prediction in delivery of healthcare to the patient. In various implementations, all of the data provided to the predictive machine learning classifier to obtain the prediction may be collected non-invasively or minimally-invasively (e.g., a blood draw for NIPS or biomarkers) during one or more routine medical visits of the patient. In various implementations, the method may further comprise using the predictive machine learning classifier to evaluate a patient during a current pregnancy by: receiving, from a health record system, data corresponding to one or more prior pregnancies of the patient, one or more health characteristics, and one or more cfDNA metrics; feeding the data to the predictive machine learning classifier to obtain a prediction for development of the preterm preeclampsia during the current pregnancy; and/or using the prediction in delivery of healthcare to the patient. In various implementations, all the data input into the predictive machine learning classifier to obtain the prediction may be collected non-invasively or minimally-invasively during one or more routine medical visits of the patient. In various implementations, the preterm preeclampsia may be an early onset preterm preeclampsia.

Various potential embodiments of the disclosed approach relate to a computing system configured to apply a predictive machine learning classifier to generate a prediction of a patient developing a preterm preeclampsia condition during a current pregnancy. The predictive machine learning classifier may comprise one or more artificial neural networks. The computing system may comprise one or more processors configured to receive, during the current pregnancy of the patient, through a communications network, patient data comprising a set of health characteristics and one or more cfDNA measurements. The one or more processors may be configured to feed the patient data to the predictive machine learning classifier to obtain a prediction of the patient developing the preterm preeclampsia during the current pregnancy. Data used for, and/or fed to, the predictive machine learning classifier may be acquired from or via one or more electronic health record systems. The one or more processors may be configured to provide the prediction for care of the patient. Providing the prediction may comprise transmitting, through the communications network, the prediction to at least one of a health record system (e.g., electronic medical record) and/or a computing device associated with a healthcare provider. The predictive machine learning classifier may have been trained by applying one or more deep learning techniques to data on subjects in a cohort of pregnant subjects. The training data may have comprised, for each pregnant subject, a plurality of (A) a first feature set indicative of one or more outcomes of one or more prior pregnancies of the pregnant subject; (B) a second feature set corresponding to one or more health indicators for the pregnant subject; and/or (C) a third feature set corresponding to one or more cell-free DNA metrics for the pregnant subject.

In various implementations, all of the patient data used to obtain the prediction may have been collected non-invasively during one or more routine medical visits of the patient. In various implementations, the patient data used to obtain the prediction may exclude ultrasound data. In various implementations, the preterm preeclampsia is an early onset preterm preeclampsia. In various implementations, the one or more processors are configured to generate the training dataset and apply the deep learning techniques to the training dataset to generate the predictive machine learning classifier. In various implementations, the one or more processors are configured to receive at least a subset of the health data from the health record system via the communications network. In various implementations, the one or more processors are configured to receive at least a subset of the health data via a software application running on the computing device associated with the healthcare provider.

Various embodiments of the disclosure may relate to processes performed using devices and/or systems disclosed herein.

Various embodiments of the disclosure may relate to computing systems and/or devices for performing processes disclosed herein.

### BRIEF DESCRIPTION OF THE DRAWINGS

FIG. 1 depicts an example system for implementing the disclosed approach, according to various potential embodiments.

FIG. 2 depicts example modeling and prediction processes, according to various potential embodiments.

FIG. 3 shows a simplified block diagram of a representative server system and client computer system usable to implement certain embodiments of the present disclosure.

FIG. 4 depicts data used to generate and validate an example model, according to potential embodiments of the disclosed approach.

FIG. 5 represents an example production workflow, according to potential embodiments of the disclosed approach.

FIG. 6 depicts feature significance and relative contribution to the final prediction of preterm preeclampsia, according to potential embodiments of the disclosed approach. The top (A) corresponds to a linear neural network (LNN) model, and the bottom (B) corresponds to a non-linear neural network (NLNN) model. Average contribution and 95% confidence intervals are shown. The magnitude and direction of the bars represent the contribution of each single feature to the definition of patients positive for preterm preeclampsia.

FIG. 7 depicts feature significance and relative contribution to the final prediction of preterm preeclampsia for the reduced models where the cfDNA features are omitted, according to potential embodiments of the disclosed approach. The top (A) corresponds to a reduced LNN model, and the bottom (B) corresponds to a reduced NLNN model. Average contribution and 95% confidence intervals are shown. The magnitude and direction of the bars represent the contribution of each single feature to the definition of patients positive for preterm preeclampsia.

The foregoing and other features of the present disclosure will become apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings.

### DETAILED DESCRIPTION

It is to be appreciated that certain aspects, modes, embodiments, variations and features of the present methods are described below in various levels of detail in order to provide a substantial understanding of the present technology. It is to be understood that the present disclosure is not limited to particular uses, methods, devices, or systems, each of which can, of course, vary. It is also to be understood that the terminology used herein is for the purpose of describing particular embodiments only, and is not intended to be limiting.

Lower-intervention, preventative treatments (such as low-dose aspirin) tend to be associated with a decreased risk of complications or adverse outcomes, as well as decreased costs, and are thus generally preferable to higher-intervention treatments. Patient characteristics known to be associated with increased risk for preeclampsia are routinely collected as part of prenatal care. Additionally, first trimester maternal and fetoplacental cell-free DNA (cfDNA) results, which may be associated with an increased risk for development of preeclampsia, are frequently available as part of non-invasive prenatal screening (NIPS), the predominant screening test for fetal chromosomal abnormalities in the US.

In potential implementations, artificial neural network models are generated and/or used for the prediction of preterm preeclampsia (preeclampsia with birth <37 weeks' gestation) and early onset preeclampsia (preeclampsia with birth <34 weeks gestation) using patient characteristics that are routinely available and/or that are non-invasively acquired, such as health data available at the first antenatal visit and data from cfDNA screenings.

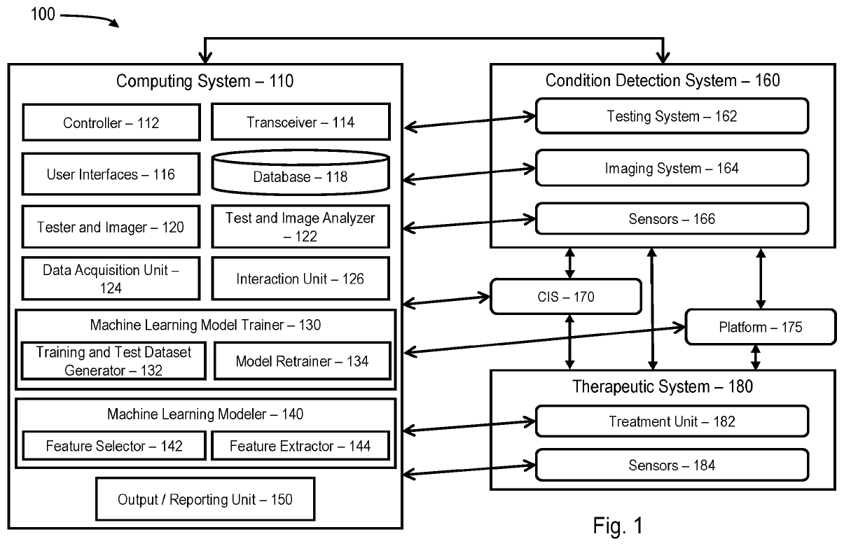

Referring to FIG. 1, in potential implementations, a system 100 may include a computing system 110 (which may be or may include one or more computing devices, co-located or remote to each other), a condition detection system 160, a Clinical Information System (CIS) 170 (used interchangeably with electronic medical record (EMR) system or electronic health record (EHR) system), a platform 175, and a therapeutic system 180. The computing system 110 (e.g., one or more computing devices) may be used to control and/or exchange signals and/or data with condition detection system 160, CIS 170, platform 175, and/or therapeutic system 180, directly or via another component of system 100. In certain embodiments, computing system 110 may be used to control and/or exchange data or other signals with condition detection system 160, CIS 170, platform 175, and/or therapeutic system 180. The computing system 110 may include one or more processors and one or more volatile and non-volatile memories for storing computing code and data that are captured, acquired, recorded, and/or generated.

The computing system 110 may include a controller 112 that is configured to exchange control signals with condition detection system 160, CIS 170, platform 175, therapeutic system 180, and/or any components thereof, allowing the computing system 110 to be used to control, for example, acquisition of patient data such as test results, capture of images, acquisition of signals by sensors, positioning or repositioning of subjects and patients, recording or obtaining other subject or patient information, and applying therapies.

A transceiver 114 allows the computing system 110 to exchange readings, control commands, and/or other data, wirelessly or via wires, directly or via networking protocols, with condition detection system 160, CIS 170, platform 175, and/or therapeutic system 180, or components thereof. One or more user interfaces 116 allow the computing device 110 to receive user inputs (e.g., via a keyboard, touchscreen, microphone, camera, etc.) and provide outputs (e.g., via a display screen, audio speakers, etc.) with users. The computing device 110 may additionally include one or more databases 118 for storing, for example, data acquired from one or more systems or devices, signals acquired via one or more sensors, biomarker signatures, etc. In some implementations, database 118 (or portions thereof) may alternatively or additionally be part of another computing device that is co-located or remote (e.g., via “cloud computing”) and in communication with computing device 110, condition detection system 160, CIS 170, platform 175, and/or therapeutic system 180 or components thereof.

Condition detection system 160 may include a testing system 162, which may be or may include, for example, any system or device that is involved in, for example, analyzing samples (e.g., blood, plasma, or urine), recording patient data, and/or obtaining laboratory or other test results (e.g., DNA data). An imaging system 164 may be any system or device used to capture imaging data, such as a magnetic resonance imaging (MRI) scanner, a positron emission tomography (PET) scanner, a single photon emission computed tomography (SPECT) scanner, a computed tomography (CT) scanner, a fluoroscopy scanner, and/or other imaging devices and/or sensors. Sensors 166 may detect, for example, a position or motion of a patient, organs, tissues, physiological readings such as lung capacity or heart activity/signals, or other states and/or conditions of the patient.

Therapeutic system 180 may include a treatment unit 180, which may be or may include, for example, a radiation source for external beam therapy (e.g., orthovoltage x-ray machines, Cobalt-60 machines, linear accelerators, proton beam machines, neutron beam machines, etc.) and/or one or more other treatment devices. Sensors 184 may be used by therapeutic system 180 to evaluate and guide a treatment (e.g., by detecting level of emitted radiation, a condition or state of the patient, or other states or conditions). In various implementations, components of system 100 may be rearranged or integrated in other configurations. For example, computing system 110 (or components thereof) may be integrated with one or more of the condition detection system 160, therapeutic system 180, and/or components thereof. The condition detection system 160, therapeutic system 180, and/or components thereof may be directed to a platform 175 on which a patient, subject, or sample can be situated (so as to test a sample, image a subject, apply a treatment or therapy to the subject, detect activity and/or motion of the subject in a stress test, etc.). In various embodiments, the platform 175 may be movable (e.g., using any combination of motors, magnets, etc.) to allow for positioning and repositioning of samples and/or subjects (such as micro-adjustments to compensate for motion of a subject or patient or to position the patient for scans of different regions of interest). The platform 175 may include its own sensors to detect a condition or state of the sample, patient, and/or subject.

The computing system 110 may include a tester and imager 120 configured to direct, for example, laboratory tests, image capture, and acquisition of test and/or imaging data. Tester and imager 120 may include an image generator that may convert or transform raw imaging data from condition detection system 160 into usable medical images or into another form to be analyzed. Computing system 110 may include a test and image analyzer 122 configured to, for example, analyze raw test results to generate relevant metrics, identify features in images or imaging data, or otherwise make use of tests or testing data, and/or images or imaging data.

A data acquisition unit 124 may retrieve, acquire, or otherwise obtain various data to be used to, for example, train models using data on subjects, or apply trained models using data on patients. The data acquisition unit 124 may, for example, obtain test data stored in CIS 170. An interaction unit 126 may interact (e.g., via user interfaces 116) with users (e.g., patients and/or healthcare providers) to obtain information needed for a model or to provide information to users. In certain embodiments, the data acquisition unit 124 may obtain data from users via interaction unit 126.

A machine learning model trainer 130 (“ML model trainer”) may be configured to train machine learning models used herein, as further discussed below. The ML model trainer may include a training dataset generator that processes various data to generate one or more training datasets to be used, for example, to train the models discussed herein. In certain embodiments, as more data and outcomes become available, a model retrainer 134 may further train a previously-trained model to improve or otherwise update or revise models. The ML model trainer may apply various machine learning techniques to the training datasets. A machine learning modeler 140 (“ML modeler”) may be configured to apply trained machine learning models to particular patient data. A feature selector 142 may be configured to select which features are to be fed to a model to obtain a prediction (e.g., a likelihood of developing term preeclampsia, preterm preeclampsia, and/or early onset preterm preeclampsia). The feature selector 142 may select features based on, for example, which features were previously employed to train the ML model(s) to be used, the parameters of the model(s), and/or how influential the features were in more predicting outcomes (e.g., feature coefficients). The feature selector 142 may additionally or alternatively select features based on what data (e.g., test results) are available for the patient. For example, if data corresponding to one feature in a “tier 1” feature set (e.g., the “top five” most influential features with the five highest coefficients) is not available for the patient, two features in a “tier 2” feature set (e.g., features with the 6th to 10th highest coefficients) may be selected instead. A feature extractor 144 may obtain values for selected features from various data sources. An outputting and reporting unit 150 may transmit models or predictions thereof (to, e.g., healthcare professionals, patients, etc.), or generate and provide reports based on the models or predictions thereof. The reports may include, for example, model outputs (e.g., a prediction) along with information on the basis for a prediction, and guidelines and/or proposed recommendations and next steps based on the predictions, so as to identify for clinicians the factors most impactful on low or high likelihoods or other prognoses and guide clinicians and patients.

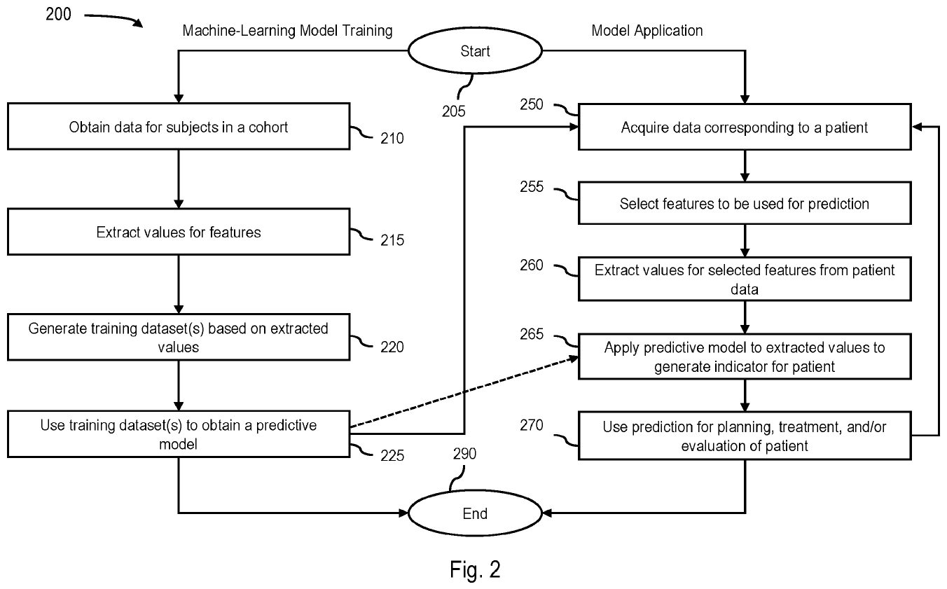

Referring to FIG. 2, an example process 200 is illustrated, according to various potential embodiments. Various elements of process 200 may be implemented by or via system 100 or components thereof. Process 200 may begin (205) with model training (on the left side of FIG. 2), which may be implemented by or via computing system 110, if a model is not already available (e.g., in database 118), or if additional models are to be generated or updated through training or retraining with new training data. Alternatively, process 200 may begin with use (application) of a model (on the right side of FIG. 2) for a patient if a trained model is already available. Predictions may be implemented by or via computing system 110 if a suitable trained model is available. In various embodiments, process 200 may comprise both model training (e.g., steps 210-225) followed by prognosis prediction (e.g., steps 250-270).

At 210, health data pertaining to subjects may be obtained. This may be obtained by or via, for example, condition detection system 160 and/or CIS 170 for a cohort of, for example, 10,000, 11,000, 12,000, 13,000, 14,000, 15,000, or more subjects. This may represent, for example, multiple years of treatments (e.g., multiple pregnancies. The health data should have enough data (e.g., across multiple pregnancies) to be able to capture and account for a variety of features and build a generalizable model. Laboratory test or imaging data may be transformed, converted, or otherwise analyzed by test and image analyzer 122. Step 215 involves extracting, from (or based on) the health data, feature values corresponding to the subjects in the cohort.

At 220, one or more datasets (e.g., training datasets) may be generated using the extracted feature values corresponding health data of the subjects. This may be performed, for example, by or via ML model trainer 130, or more specifically, training and test dataset generator 132. At 225, the one or more datasets may be used to train and test a model for making predictions. The model may be stored (e.g., in database 118) for subsequent use. Process 200 may end (290), or may proceed to step 250 for use in prognosis prediction. (As represented by the dotted line from step 225 to step 265, the model may subsequently be used to generate and use predictions.)

At 250, health data of a patient may be obtained and analyzed (e.g., by or via condition detection system 160 and/or CIS 170). Test data may correspond to various laboratory tests performed on samples of the patient. At 255, a set of features may be selected (e.g., by or via feature selector 142), and at 260, values for the features may be extracted from the health data of the patient (e.g., by or via feature extractor 144).

At 265, feature values extracted from patient health data may be input to a predictive model (e.g., a machine learning classifier) to predict patient prognosis. At 270, the predicted prognosis and the factors underlying the prognosis may be used in caring for the patient. For example, predicted prognosis may be used for planning for a potential outcome and/or identifying potentially preventative care. Additionally or alternatively, predicted prognosis can be used in evaluation of a patient's health and changes or trends therein, such as whether the patient is deteriorating or improving as indicated by changes in prognoses over time from the ML modeler as new tests are run or otherwise as new health data becomes available.

Process 200 may end (290), or return to step 250 (e.g., after running another test or administering a treatment) for subsequent planning based on a change in a condition of the patient.

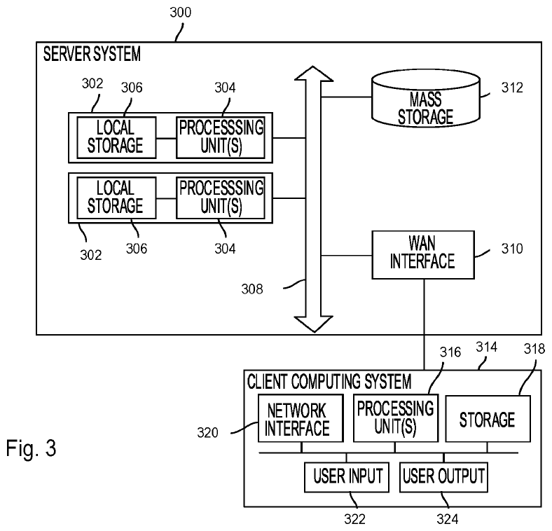

Various operations described herein can be implemented on computer systems having various configurations. FIG. 3 shows a simplified block diagram of a representative server system 300 (e.g., computing system 110) and client computer system 314 (e.g., computing system 110, condition detection system 160, CIS 170, and/or therapeutic system 180) usable to implement various embodiments of the present disclosure. In various embodiments, server system 300 or similar systems can implement services or servers described herein or portions thereof. Client computer system 314 or similar systems can implement clients described herein.

Server system 300 can have a modular design that incorporates a number of modules 302 (e.g., blades in a blade server embodiment); while two modules 302 are shown, any number can be provided. Each module 302 can include processing unit(s) 304 and local storage 306.

Processing unit(s) 304 can include a single processor, which can have one or more cores, or multiple processors. In some embodiments, processing unit(s) 304 can include a general-purpose primary processor as well as one or more special-purpose co-processors such as graphics processors, digital signal processors, or the like. In some embodiments, some or all processing units 304 can be implemented using customized circuits, such as application specific integrated circuits (ASICs) or field programmable gate arrays (FPGAs). In some embodiments, such integrated circuits execute instructions that are stored on the circuit itself. In other embodiments, processing unit(s) 304 can execute instructions stored in local storage 306. Any type of processors in any combination can be included in processing unit(s) 304.

Local storage 306 can include volatile storage media (e.g., conventional DRAM, SRAM, SDRAM, or the like) and/or non-volatile storage media (e.g., magnetic or optical disk, flash memory, or the like). Storage media incorporated in local storage 306 can be fixed, removable or upgradeable as desired. Local storage 306 can be physically or logically divided into various subunits such as a system memory, a read-only memory (ROM), and a permanent storage device. The system memory can be a read-and-write memory device or a volatile read-and-write memory, such as dynamic random-access memory. The system memory can store some or all of the instructions and data that processing unit(s) 304 need at runtime. The ROM can store static data and instructions that are needed by processing unit(s) 304. The permanent storage device can be a non-volatile read-and-write memory device that can store instructions and data even when module 302 is powered down. The term “storage medium” as used herein includes any medium in which data can be stored indefinitely (subject to overwriting, electrical disturbance, power loss, or the like) and does not include carrier waves and transitory electronic signals propagating wirelessly or over wired connections.

In some embodiments, local storage 306 can store one or more software programs to be executed by processing unit(s) 304, such as an operating system and/or programs implementing various server functions or any system or device described herein.

“Software” refers generally to sequences of instructions that, when executed by processing unit(s) 304 cause server system 300 (or portions thereof) to perform various operations, thus defining one or more specific machine embodiments that execute and perform the operations of the software programs. The instructions can be stored as firmware residing in read-only memory and/or program code stored in non-volatile storage media that can be read into volatile working memory for execution by processing unit(s) 304. Software can be implemented as a single program or a collection of separate programs or program modules that interact as desired. From local storage 306 (or non-local storage described below), processing unit(s) 304 can retrieve program instructions to execute and data to process in order to execute various operations described above.

In some server systems 300, multiple modules 302 can be interconnected via a bus or other interconnect 308, forming a local area network that supports communication between modules 302 and other components of server system 300. Interconnect 308 can be implemented using various technologies including server racks, hubs, routers, etc.

A wide area network (WAN) interface 310 can provide data communication capability between the local area network (interconnect 308) and a larger network, such as the Internet. Conventional or other activities technologies can be used, including wired (e.g., Ethernet, IEEE 802.3 standards) and/or wireless technologies (e.g., Wi-Fi, IEEE 802.11 standards).

In some embodiments, local storage 306 is intended to provide working memory for processing unit(s) 304, providing fast access to programs and/or data to be processed while reducing traffic on interconnect 308. Storage for larger quantities of data can be provided on the local area network by one or more mass storage subsystems 312 that can be connected to interconnect 308. Mass storage subsystem 312 can be based on magnetic, optical, semiconductor, or other data storage media. Direct attached storage, storage area networks, network-attached storage, and the like can be used. Any data stores or other collections of data described herein as being produced, consumed, or maintained by a service or server can be stored in mass storage subsystem 312. In some embodiments, additional data storage resources may be accessible via WAN interface 310 (potentially with increased latency).

Server system 300 can operate in response to requests received via WAN interface 310. For example, one of modules 302 can implement a supervisory function and assign discrete tasks to other modules 302 in response to received requests. Conventional work allocation techniques can be used. As requests are processed, results can be returned to the requester via WAN interface 310. Such operation can generally be automated. Further, in some embodiments, WAN interface 310 can connect multiple server systems 300 to each other, providing scalable systems capable of managing high volumes of activity. Conventional or other techniques for managing server systems and server farms (collections of server systems that cooperate) can be used, including dynamic resource allocation and reallocation.

Server system 300 can interact with various user-owned or user-operated devices via a wide-area network such as the Internet. An example of a user-operated device is shown in FIG. 3 as client computing system 314. Client computing system 314 can be implemented, for example, as a consumer device such as a smartphone, other mobile phone, tablet computer, wearable computing device (e.g., smart watch, eyeglasses), desktop computer, laptop computer, and so on.

Client computing system 314 can communicate via WAN interface 310. Client computing system 314 can include conventional computer components such as processing unit(s) 316, storage device 318, network interface 320, user input device 322, and user output device 324. Client computing system 314 can be a computing device implemented in a variety of form factors, such as a desktop computer, laptop computer, tablet computer, smartphone, other mobile computing device, wearable computing device, or the like.

Processor 316 and storage device 318 can be similar to processing unit(s) 304 and local storage 306 described above. Suitable devices can be selected based on the demands to be placed on client computing system 314; for example, client computing system 314 can be implemented as a “thin” client with limited processing capability or as a high-powered computing device. Client computing system 314 can be provisioned with program code executable by processing unit(s) 316 to enable various interactions with server system 300 of a message management service such as accessing messages, performing actions on messages, and other interactions described above. Some client computing systems 314 can also interact with a messaging service independently of the message management service.

Network interface 320 can provide a connection to a wide area network (e.g., the Internet) to which WAN interface 310 of server system 300 is also connected. In various embodiments, network interface 320 can include a wired interface (e.g., Ethernet) and/or a wireless interface implementing various RF data communication standards such as Wi-Fi, Bluetooth, or cellular data network standards (e.g., 3G, 4G, 5G, LTE, etc.).

User input device 322 can include any device (or devices) via which a user can provide signals to client computing system 314; client computing system 314 can interpret the signals as indicative of particular user requests or information. In various embodiments, user input device 322 can include any or all of a keyboard, touch pad, touch screen, mouse or other pointing device, scroll wheel, click wheel, dial, button, switch, keypad, microphone, and so on.

User output device 324 can include any device via which client computing system 314 can provide information to a user. For example, user output device 324 can include a display to display images generated by or delivered to client computing system 314. The display can incorporate various image generation technologies, e.g., a liquid crystal display (LCD), light-emitting diode (LED) including organic light-emitting diodes (OLED), projection system, cathode ray tube (CRT), or the like, together with supporting electronics (e.g., digital-to-analog or analog-to-digital converters, signal processors, or the like). Some embodiments can include a device such as a touchscreen that function as both input and output device. In some embodiments, other user output devices 324 can be provided in addition to or instead of a display. Examples include indicator lights, speakers, tactile “display” devices, printers, and so on.

Some embodiments include electronic components, such as microprocessors, storage and memory that store computer program instructions in a computer readable storage medium. Many of the features described in this specification can be implemented as processes that are specified as a set of program instructions encoded on a computer readable storage medium. When these program instructions are executed by one or more processing units, they cause the processing unit(s) to perform various operation indicated in the program instructions. Examples of program instructions or computer code include machine code, such as is produced by a compiler, and files including higher-level code that are executed by a computer, an electronic component, or a microprocessor using an interpreter. Through suitable programming, processing unit(s) 304 and 316 can provide various functionality for server system 300 and client computing system 314, including any of the functionality described herein as being performed by a server or client, or other functionality associated with message management services.

It will be appreciated that server system 300 and client computing system 314 are illustrative and that variations and modifications are possible. Computer systems used in connection with embodiments of the present disclosure can have other capabilities not specifically described here. Further, while server system 300 and client computing system 314 are described with reference to particular blocks, it is to be understood that these blocks are defined for convenience of description and are not intended to imply a particular physical arrangement of component parts. For instance, different blocks can be but need not be located in the same facility, in the same server rack, or on the same motherboard. Further, the blocks need not correspond to physically distinct components. Blocks can be configured to perform various operations, e.g., by programming a processor or providing appropriate control circuitry, and various blocks might or might not be reconfigurable depending on how the initial configuration is obtained. Embodiments of the present disclosure can be realized in a variety of apparatus including electronic devices implemented using any combination of circuitry and software.

### Example Methods

Analysis: A secondary analysis of the Single-nucleotide-polymorphism (SNP)-based Microdeletion and Aneuploidy RegisTry (SMART) was performed. SMART was designed to evaluate the performance of SNP-based NIPS in a general pregnant population undergoing screening as part of their clinical care. Twenty-one sites in 6 countries participated in the study between April, 2015 and January, 2019. The study was restricted to singleton pregnancies, ≥9 weeks' gestation, and with maternal age 218 years. Data on patient characteristics, NIPS results, serum screening results, ultrasound, and pregnancy outcomes were collected by local research coordinators.

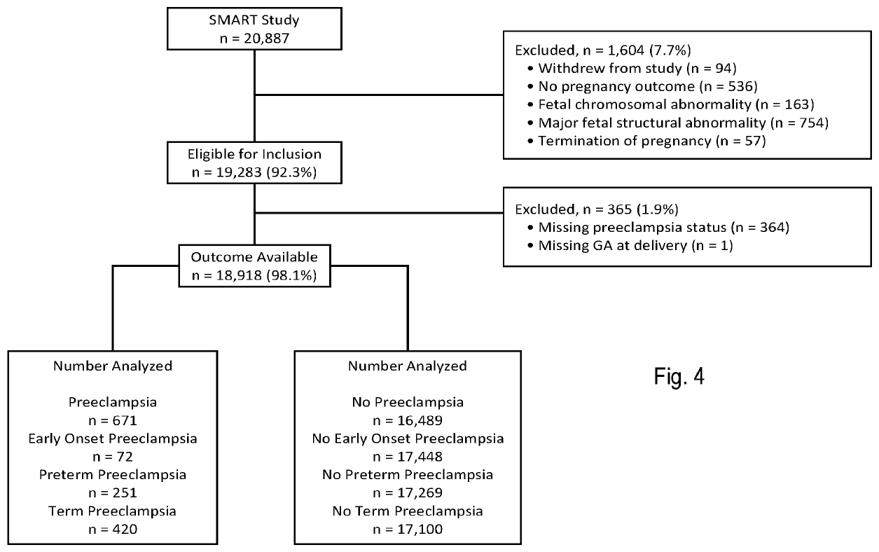

Study Population: With reference to FIG. 4, a total of 20,887 women participated in SMART. For this analysis, pregnancies were excluded if there was a fetal chromosomal or major structural abnormality, termination of pregnancy, missing data on pregnancy outcome, participant withdrawal from the study (n=1604), or if there were missing data on preeclampsia status or gestational age at delivery (n=365). Participants were further excluded if they had missing data for any of the variables required for model development. The final cohort used in model development comprised 17,520 participants: 671 with preeclampsia and 16,849 without preeclampsia. Of those with preeclampsia, 251 had preterm preeclampsia, 72 had early onset preeclampsia, and 420 had term preeclampsia. Preeclampsia is a pregnancy-associated condition that consists of hypertension with or without evidence of organ dysfunction (e.g., liver and kidney dysfunction), symptoms (e.g., headache and visual disturbance), and in severe cases, seizures (“eclampsia”). The presence of “preeclampsia” was based on the presence of a clinical diagnosis of preeclampsia documented in the participant's medical record.

Patient Characteristics: Patient characteristics examined in the analysis included maternal age, body mass index (BMI), maternal height, maternal weight, country, race/ethnicity, parity, use of in-vitro fertilization, chronic hypertension, pre-pregnancy diabetes, and cigarette smoking during pregnancy. Features obtained from SNP-based NIPS comprised total cfDNA and fetal fraction (FF). Patient characteristics were compared between the groups of patients with and without preeclampsia. Comparison of continuous data was performed using the Wilcoxon Rank Sum test and comparison of independent categorical data was performed using the Chi-squared test. P-values were adjusted for multiple comparisons using the Holm method.

Outcome Measures: The primary outcome was preterm preeclampsia defined as preeclampsia with birth <37 weeks' gestation. Secondary outcomes were early onset preeclampsia (preeclampsia with birth <34 weeks' gestation) and term preeclampsia (preeclampsia with birth >37 weeks' gestation).

Features in the model: Variables input into the predictive models included 13 patient characteristics available in the SMART study data and identified as risk factors for developing preeclampsia based on the existing medical literature (Table 1). Two example features obtained from SNP-based cfDNA screening (total cTDNA and FF) were also included. FF was estimated through a probability model using SNPs that were deemed homozygous in the maternal alleles. The probability model employed a maximum likelihood approach, based on the observed minor allele frequencies, to determine the fetal fraction value that maximizes the likelihood function. Two features obtained from SNP-based cPDNA screening, total cfDNA (maternal and fetal) and FF, were also included. Six of the 15 features were encoded as continuous variables; the remaining 10 features were ncoded as binary (Table 1).

TABLE 1
Description, encoded name and type of the input variables used
in the predictive models. Description with encoded name

| | Encoded | Encoded |
|---|---|---|
| Description | Name | Type |
| Fetal fraction | FF | Continuous |
| Total cell-free DNA | Total cfDNA | Continuous |
| Maternal age (years) | MATAGE | Continuous |
| Maternal height (cm) | height_cm | Continuous |
| Maternal weight (kg) | weight_kg | Continuous |
| Gestational age at cfDNA screening | PANGA | Continuous |
| Pregnancy conceived using in vitro | IVF | Binary [0, 1] |
| fertilization | | |
| Cigarette smoking during pregnancy | SMOKEPREG | Binary [0, 1] |
| Parity: nulliparous vs multiparous | PARITY | Binary [0, 1] |
| Pre-pregnancy hypertension | BHXHTN | Binary [0, 1] |
| Pre-pregnancy diabetes | BDIABTYP_PRE | Binary [0, 1] |
| Previous preterm birth (<37 weeks' | PREVPTB | Binary [0, 1] |
| gestation) | | |
| Previous stillbirth | PREVSTILL | Binary [0, 1] |
| Previous cesarean birth | PREVCS | Binary [0, 1] |
| Recurrent pregnancy loss (≥3 losses | RECURLOSS | Binary [0, 1] |
| at <20 weeks' gestation | | |

Including race and ethnicity in predictive modeling has the potential to perpetuate racial inequities. Race and ethnicity were therefore not included in model development for this example embodiment. However, given recent research supporting the importance of race in preeclampsia prediction a sensitivity analysis was performed to evaluate the effect of including race/ethnicity on the predictive accuracy of the model. Ethnicity information was added as a binary flag to the feature set in Table 1. Four ethnic groups (Black, Asian, Latina, and White) were modeled separately using the neural network model development and the cross-validation protocol described in this Methods section. For example, for the Black group, a binary flag was added to the feature list in Table 1. This flag was set to one for the samples listed as of “Black” ethnicity and zero for every other sample. Each group was sequentially explored in this way, and the area under the receiver operating characteristic (AUROC) performance metric was calculated for each iteration. In some implementations, models may be trained and tested (validated), for example, using a common protocol based on stratified, repeated k-fold cross validation (SRKFCV).

Machine Learning Models for Classification: First, a linear neural network (LNN) model was considered as a “reference classifier.” The LNN model is a neural network architecture that directly connects input features to a single sigmoid output neuron. In addition, a number of feedforward, dense, neural networks models (NLNN models) were considered, incrementally adding one or more hidden layers of rectified linear units (reLu) units to the LNN model. The performance of the classifiers was evaluated on a number of metrics: AUC score, sensitivity, positive predictive value and negative predictive value at different values for the screen positive rate metric.

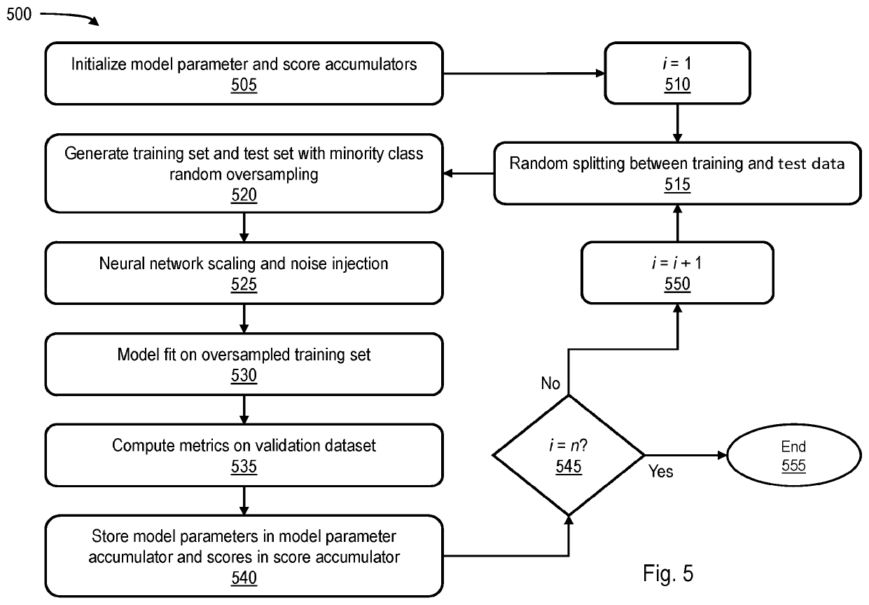

Training and Internal Validation: With reference to FIG. 5, an example training and validation process 500 includes a series of steps that are discussed below. All neural network models in the example implementations discussed with respect to FIG. 5 below were implemented and optimized in keras.tensorflow using the binary cross entropy as the loss function. The network's parameters were optimized via stochastic gradient descent, with a constant learning rate of 1e-2 and zero momentum.

At block 505, a model parameter accumulator and a score accumulator initialized. The model parameter accumulator and the score accumulator are data structures in which results are stored at each iteration of the loop discussed below (blocks 515 to 550). At block 510, a variable i is initialized to indicate the iteration (here, first iteration) of a series of steps that may loop until a value (n) is reached.

At block 515, there is a stratified split, in which the dataset was randomly split into train and validation datasets. In an example implementation, the split yields the proportions of 0.9 and 0.1 corresponding to train and validation, respectively (i.e., 90% for training and 10% for testing). Here, the random split was also stratified with regards to the outcome.

At block 520, a training dataset and a test dataset were generated, with steps taken to account for unbalanced data. Of the study population, only 1.4% developed preterm preeclampsia, hence a machine learning model applied to such a dataset would typically be expected to over-predict the majority class (i.e., patients without preterm preeclampsia) and be relatively inefficient in predicting the “rare” events associated with the minority class (i.e., patients who developed preterm preeclampsia). In light of this, oversampling was applied to the positive samples in the training set, after the train and validation split (515), via uniform random sampling with replacement, yielding a training set with an equal number of positive and negative samples.

At block 525, example process 500 includes a step of neural network scaling and noise injection. A standard scaler was fitted against the oversampled training dataset to appropriately scale the train and validation datasets. Gaussian noise with mean equal to zero and standard deviation equal to 1e-1 was added to the standardized training dataset. The standard scaler and the Gaussian noise were only applied to the continuous input features (Table 1). Noise injection was used both as a form of regularization and data augmentation.

At block 530, the model was fit on the oversampled training dataset. The fitting procedure was then performed on 100 epochs and a mini batch size of 32 using stochastic gradient descent with a constant learning rate of 1e-2.

At block 535, relevant performance metrics were calculated on the validation dataset. Specifically, the predictive performance of the two classifiers was evaluated by calculating the area under the receiver-operating characteristic (AUC) curve in addition to sensitivity, positive predictive value, and negative predictive value for fixed screen positive rates.

At block 540, model parameters were stored in the model parameters accumulator, and scores were stored in the score accumulator. To repeat steps in a loop n number of times, at 545, it is determined whether i (initially equal to 1) is equal to n. If not (e.g., if i<n), at block 550, i is incremented upwards, and process 500 loops back to block 515. If so (e.g., if i=n), process 500 ends at block 555.

Internal Validation: To minimize or otherwise reduce the bias intrinsic to a single instance of the random split at block 515, blocks 515 through 530 were repeated 200 times (n=200) in example process 500. This Monte Carlo cross validation generated 200 estimations for each of the performance metrics of interest, corresponding to 200 different random training-validation splits. As a result, the collected metrics are de-facto stochastic variables and can be analyzed as such. For each metric the mean value and its 95% confidence interval were considered to evaluate the “expected behavior” of the predictive models.

Network Regularization: In addition to the noise injection described above (block 525), regularization was also introduced in the example models via the kernel_regularizer using a L2 penalty whose magnitude varied between 0.0 and 1e-2.

Hyperparameter Optimization: Additionally, an incremental, although partial, grid-based hyperparameter search was performed, first considering and fixing the number of epochs and the batch size. Second, hidden layers number and size were explored, as well as three regularization methods: L2 penalty magnitude in kernel regularization, percentage in layer dropout, and standard deviation in Gaussian noise injection in the training dataset.

Model Performance: Model performance was assessed by the expected area under the curve (AUC) score and the sensitivity for fixed screen positive rates 10%, 15%, and 20% for the primary and secondary outcomes.

Characteristics of the study population: With reference to FIG. 4 again, of 17,520 participants included in the final analysis, 671 (3.8%) developed preeclampsia. Of them, 251 (1.4%) developed preterm preeclampsia, 72 (0.4%) early onset preeclampsia, and 420 (2.4%) term preeclampsia. Characteristics of the 17,520 participants (671 with preeclampsia and 16,849 without preeclampsia) included in model development are shown in Table 2. The group that developed preeclampsia had a similar maternal age distribution to those who did not develop preeclampsia (34.3 versus 34.7 years; p=0.022). The proportion of participants with a BMI over 30 kg/m² was significantly higher in the preeclampsia group (40.8% versus 18.8%; p<0.001). Participants in the USA, participants who were nulliparous, Black, or had a history of preterm birth, stillbirth, cesarean delivery, chronic hypertension or pre-pregnancy diabetes, were overrepresented in the group that developed preeclampsia. Median gestational age at cfDNA measurement was 12.6 weeks and n=16,653 (95.1%) of patients had their cfDNA measurement at <20 weeks of gestation. cfDNA parameters were also significantly different in the group that developed preeclampsia. Total cfDNA was significantly higher (median of 362.3 DNA copies/ml versus 339.0 DNA copies/ml; p<0.001) and FF significantly lower (median of 7.5% versus 9.4%; p<0.001) in the preeclampsia group (Table 2).

TABLE 2
Baseline demographic and clinical characteristics by preeclampsia
for participants with complete data (modeling cohort)

| | preeclampsia | | No preeclampsia | | P | Adj P* |
|---|---|---|---|---|---|---|
| n (%) | 671 | (3.8%) | 16849 | (96.2%) | | |
| Maternal Age | | | | | 0.022 | 0.109 |
| Median (25% ile, 75% ile) | 34.3 | (29, 37.9) | 34.7 | (30.7, 37.5) | | |
| <20 years | 14 | (2.1%) | 215 | (1.3%) | | |
| 20-34 years | 309 | (46.1%) | 7317 | (43.4%) | | |
| ≥35 years | 301 | (44.9%) | 8095 | (48.0%) | | |
| BMI | | | | | <0.001 | <0.001 |
| Median (25% ile, 75% ile) | 28.7 | (24.3, 33.5) | 24.7 | (22.2, 28.5) | | |
| Underweight <18.5 | 8 | (1.2%) | 273 | (1.6%) | | |
| Normal 18.5-24.9 | 184 | (27.4%) | 8459 | (50.2%) | | |
| Overweight 25.0-29.9 | 197 | (29.4%) | 4720 | (28.0%) | | |
| Class I Obesity 30.0-34.9 | 138 | (20.6%) | 1929 | (11.4%) | | |
| Class II Obesity 35.0-39.9 | 67 | (10%) | 783 | (4.6%) | | |
| Class III Obesity ≥40.0 | 69 | (10.3%) | 448 | (2.7%) | | |
| Height (cm) | | | | | <0.001 | <0.001 |
| Median (25% ile, 75% ile) | 162.6 | (157.5, 167.6) | 165 | (160, 170) | | |
| Weight (kg) | | | | | <0.001 | <0.001 |
| Median (25% ile, 75% ile) | 73.9 | (63.7, 89.3) | 66.7 | (59.4, 76.2) | | |
| Country | | | | | <0.001 | <0.001 |
| United States | 502 | (74.8%) | 8620 | (51.2%) | | |
| Sweden | 81 | (12.1%) | 3056 | (18.1%) | | |
| Ireland | 70 | (10.4%) | 3875 | (23%) | | |
| England | 8 | (1.2%) | 625 | (3.7%) | | |
| Australia | 8 | (1.2%) | 422 | (2.5%) | | |
| Spain | 2 | (0.3%) | 251 | (1.5%) | | |
| Race/Ethnicity | | | | | <0.001 | <0.001 |
| Asian | 51 | (7.6%) | 1441 | (8.6%) | | |
| Black | 135 | (20.1%) | 1253 | (7.4%) | | |
| White | 274 | (40.8%) | 10939 | (64.9%) | | |
| Latina | 187 | (27.9%) | 2665 | (15.8%) | | |
| Other | 15 | (2.2%) | 376 | (2.2%) | | |
| Unknown/not reported | 9 | (1.3%) | 175 | (1%) | | |
| Parity | | | | | <0.001 | <0.001 |
| Nulliparous | 373 | (55.6%) | 7301 | (43.3%) | | |
| Multiparous | 298 | (44.4%) | 9548 | (56.7%) | | |
| Previous PTB | 80 | (26.8%) | 759 | (7.9%) | <0.001 | <0.001 |
| Previous stillbirth | 21 | (7%) | 269 | (2.8%) | 0.023 | 0.109 |
| Previous cesarean birth | 134 | (45%) | 2849 | (29.8%) | <0.001 | 0.002 |
| Recurrent pregnancy loss (≥3 before | 20 | (3%) | 558 | (3.3%) | 0.718 | 1 |
| 20 w) | | | | | | |
| IVF | | | | | 0.374 | 1 |
| Yes | 42 | (6.3%) | 908 | (5.4%) | | |
| Pre-pregnancy health | | | | | | |
| Chronic hypertension | 125 | (18.6%) | 604 | (3.6%) | <0.001 | <0.001 |
| Pre-pregnancy diabetes | 43 | (6.4%) | 155 | (0.9%) | <0.001 | <0.001 |
| Cigarette smoking during pregnancy | 13 | (1.9%) | 306 | (1.8%) | 0.934 | 1 |
| Fetal Fraction | | | | | <0.001 | <0.001 |
| Median (25% ile, 75% ile) | 7.5 | (5.7, 10.6) | 9.4 | (7, 12.3) | | |
| Fetal Fraction Percentile | | | | | <0.001 | <0.001 |
| Median (25% ile, 75% ile) | 45.66 | (22.90, 73.33) | 56.62 | (31.21, 78.26) | | |
| Total cfDNA DNA copies/ml | | | | | <0.001 | <0.001 |
| Median (25% ile, 75% ile) | 362.3 | (290.2, 463) | 339 | (275.8, 422.7) | | |
| Fetal cfDNA | | | | | <0.001 | <0.001 |
| Median (25% ile, 75% ile) | 28.4 | (20.7, 40.6) | 31.4 | (23.7, 42) | | |
| Maternal cfDNA | | | | | <0.001 | <0.001 |
| Median (25% ile, 75% ile) | 333 | (261.3, 431.3) | 305.4 | (244.6, 386.4) | | |
| Serum Screening | | | | | 0.004 | 0.025 |
| Yes | 238 | (35.5%) | 5086 | (30.2%) | | |
| Gestational Age at Panorama (weeks) | | | | | <0.001 | <0.001 |
| Median (25% ile, 75% ile) | 12.7 | (11.9, 14.6) | 12.6 | (11.4, 13.7) | | |

Model selection and performance: Example implementations analyzed and optimized a relatively large number of dense, feedforward neural networks NLNN models using the protocol described above. In more detail, NLNN models with the number of hidden layers varying between 1 and 5 and the number of neurons varying between 2 and 1024 were considered. On those NLNN models, example implementations tested different regularization strategies such as L2 kernel regularization, dropout layers, and noise injection in the training set. In some example implementations, NLNN models performed similarly to linear neural network models.

In example implementations, a NLNN model may be a neural network with one hidden layer made up by 15 reLU units (resulting in a hidden layer with same dimension as the input) and with L2 regularization implemented as keras' kernel_regularizer.

The expected AUC score and the sensitivity for fixed screen positive rates 10%, 15%, and 20% for each of the outcomes are given in Table 3. The numbers shown are mean values obtained from the optimization protocol described in the Methods and represent the expected behavior of the model on unseen data. The LNN and NLNN model classifiers had similar predictive performance across each of the preeclampsia outcomes (early onset, preterm, and term preeclampsia). The AUC scores for preterm, early onset, and term preeclampsia were 0.80, 0.79, and 0.71 respectively for the NLNN model and 0.80, 0.78, and 0.71 respectively for the LNN model (Table 3). Sensitivity was highest for the primary outcome, preterm preeclampsia with the NLNN model predicting 59% of positive cases and the LNN model predicting 58% of positive cases at a screen positive rate of 15% (Table 3). In certain implementations, no differences were observed in predictive performance between the LNN and NLNN models for the secondary outcomes (Tables 4 and 5).

TABLE 3
Sensitivity and AUC score and 95% confidence intervals
for both the LNN (Linear Neural Network) and NLNN (Non
Linear Neural Network) models for predicting preterm
preeclampsia (<37 weeks), early onset preeclampsia
(<34 weeks), and term preeclampsia (>=37 weeks)

| | |
|---|---|
| LNN | NLNN |

Preterm preeclampsia (<37 weeks)

| | | |
|---|---|---|
| AUC Score (95% CI) | 0.801 | 0.800 |
| | (0.795, 0.807) | (0.794, 0.806) |
| Sensitivity (95% CI) | | |
| 10% screen pos rate | 0.506 | 0.503 |
| | (0.491, 0.521) | (0.488, 0.518) |
| 15% screen pos rate | 0.584 | 0.593 |
| | (0.569, 0.599) | (0.578, 0.608) |
| 20% screen pos rate | 0.669 | 0.677 |
| | (0.654, 0.684) | (0.662, 0.692) |

Early Onset preeclampsia (<34 weeks)

| | | |
|---|---|---|
| AUC Score (95% CI) | 0.782 | 0.797 |
| | (0.768, 0.796) | (0.783, 0.811) |
| Sensitivity (95% CI) | | |
| 10% screen pos rate | 0.475 | 0.486 |
| | (0.445, 0.505) | (0.456, 0.516) |
| 15% screen pos rate | 0.545 | 0.549 |
| | (0.525, 0.565) | (0.529,0.569) |
| 20% screen pos rate | 0.621 | 0.651 |
| | (0.601, 0.641) | (0.631, 0.671) |

Term preeclampsia (≥37 weeks)

| | | |
|---|---|---|
| AUC Score (95% CI) | 0.712 | 0.713 |
| | (0.707, 0.717) | (0.708, 0.718) |
| Sensitivity (95% CI) | | |
| 10% screen pos rate | 0.293 | 0.307 |
| | (0.283, 0.303) | (0.297, 0.317) |
| 15% screen pos rate | 0.408 | 0.417 |
| | (0.398, 0.418) | (0.407, 0.427) |
| 20% screen pos rate | 0.490 | 0.499 |
| | (0.480, 0.500) | (0.489, 0.509) |

TABLE 4
Positive predictive value (PPV) and 95% confidence intervals
for both the LNN (Linear Neural Network) and NLNN (Non Linear
Neural Network) models for predicting preterm preeclampsia (<37
weeks), early onset preeclampsia (<34 weeks), and term preeclampsia
(>=37 weeks). The prevalence was 0.015, 0.005 and 0.04
for preterm, early onset and term preeclampsia, respectively.

| | |
|---|---|
| LNN | NLNN |

Preterm preeclampsia (<37 weeks)

| | | |
|---|---|---|
| PPV (95% CI) | | |
| 10% screen pos rate | 0.074 | 0.073 |
| | (0.072, 0.076) | (0.071, 0.075) |
| 15% screen pos rate | 0.058 | 0.058 |
| | (0.056, 0.060) | (0.056, 0.060) |
| 20% screen pos rate | 0.050 | 0.050 |
| | (0.048, 0.052) | (0.048, 0.052) |

Early Onset preeclampsia (<34 weeks)

| | | |
|---|---|---|
| PPV (95% CI) | | |
| 10% screen pos rate | 0.023 | 0.023 |
| | (0.021, 0.025) | (0.021, 0.025) |
| 15% screen pos rate | 0.018 | 0.018 |
| | (0.016,0.020) | (0.016,0.020) |
| 20% screen pos rate | 0.016 | 0.016 |
| | (0.014, 0.018) | (0.014, 0.018) |

Term preeclampsia (>37 weeks)

| | | |
|---|---|---|
| PPV (95% CI) | | |
| 10% screen pos rate | 0.115 | 0.116 |
| | (0.113, 0.117) | (0.114, 0.118) |
| 15% screen pos rate | 0.106 | 0.105 |
| | (0.104, 0.108) | (0.103, 0.107) |
| 20% screen pos rate | 0.092 | 0.093 |
| | (0.090, 0.094) | (0.091, 0.095) |

TABLE 5
Negative predictive value from both the LNN (Linear Neural
Network) and NLNN (Non Linear Neural Network) models for
predicting the primary outcome (preterm preeclampsia) and
secondary outcomes (early onset preeclampsia and term preeclampsia).
The prevalence was 0.015, 0.005 and 0.04 for preterm, early
onset and term preeclampsia, respectively.

| | |
|---|---|
| LNN | NLNN |

Preterm preeclampsia (<37 weeks)

| | | |
|---|---|---|
| NPV (95% CI) | | |
| 10% screen pos rate | 0.992 | 0.992 |
| | (0.990, 0.992) | (0.990, 0.992) |
| 15% screen pos rate | 0.993 | 0.993 |
| | (0.991, 0.993) | (0.991, 0.993) |
| 20% screen pos rate | 0.994 | 0.994 |
| | (0.993, 0.995) | (0.993, 0.995) |

Early Onset preeclampsia (<34 weeks)

| | | |
|---|---|---|
| NPV (95% CI) | | |
| 10% screen pos rate | 0.997 | 0.997 |
| | (0.995, 0.999) | (0.995, 0.999) |
| 15% screen pos rate | 0.997 | 0.997 |
| | (0.985, 0.998) | (0.988, 0.996) |
| 20% screen pos rate | 0.998 | 0.998 |
| | (0.996, 1.000) | (0.996, 1.000) |

Term preeclampsia (≥37 weeks)

| | | |
|---|---|---|
| NPV (95% CI) | | |
| 10% screen pos rate | 0.969 | 0.969 |
| | (0.968, 0.970) | (0.968, 0.970) |
| 15% screen pos rate | 0.972 | 0.972 |
| | (0.971, 0.973) | (0.971, 0.973) |
| 20% screen pos rate | 0.973 | 0.974 |
| | (0.972, 0.974) | (0.973, 0.975) |

To assess the contribution of the cfDNA features to the performance of the example models in various implementations, additional training-validation runs were performed on a reduced version of the dataset where the FF and total cfDNA features were omitted. In these neural network models, the number of units in the hidden layer were rescaled to 13, to match the dimensionality of the input layer. In example implementations, for early onset preeclampsia, removal of the total cfDNA and FF features from the model was associated with an approximately 7% decrease in sensitivity at a 15% screen positive rate for both the LNN and NLNN models (Table 6).

TABLE 6
Model performance for the LNN and NLNN models with and without cfDNA features.
The models where the cfDNA features are omitted are named as “Reduced”.
“Full” refers to the models trained and optimized on the full 15 features in Table 2.

| | | | |
|---|---|---|---|
| Full LNN | Full NLNN | Reduced LNN | Reduced NLNN |

Preterm preeclampsia (<37 weeks)

| | | | | |
|---|---|---|---|---|
| AUC Score | 0.801 | 0.800 | 0.800 | 0.802 |
| | (0.795, 0.807) | (0.794, 0.806) | (0.794-0.806) | (0.798-0.808) |
| Sensitivity for 15% | 0.584 | 0.593 | 0.590 | 0.582 |
| screen positive rate | (0.569, 0.599) | (0.578, 0.608) | (0.575-0.605) | (0.567-0.597) |

Early Onset preeclampsia (<34 weeks)

| | | | | |
|---|---|---|---|---|
| AUC Score | 0.782 | 0.797 | 0.768 | 0.768 |
| | (0.768, 0.796) | (0.783, 0.811) | (0.755-0.781) | (0.755-0.781) |
| Sensitivity for 15% | 0.545 | 0.549 | 0.474 | 0.480 |
| screen positive rate | (0.525, 0.565) | (0.529,0.569) | (0.444-0.504) | (0.450-0.510) |

Term preeclampsia (≥37 weeks)

| | | | | |
|---|---|---|---|---|
| AUC Score | 0.705 | 0.706 | 0.704 | 0.707 |
| | (0.702, 0.708) | (0.703-0.709) | (0.699-0.709) | (0.702-0.712) |
| Sensitivity for 15% | 0.408 | 0.417 | 0.398 | 0.413 |
| screen positive rate | (0.398, 0.418) | (0.407, 0.427) | (0.388-0.408) | (0.403-0.423) |

The contribution of ethnicity-related information was evaluated via an additional set of models (see Methods for details), estimating the AUC score and its 95% confidence interval in example implementations (the results are listed in Table 7). After comparing these results with the ones listed in Tables 3, 4, and 5 (where ethnicity is not considered), ethnicity-related information does not seem to contribute significantly to the predictive performance of the models.

TABLE 7
Expected AUC Score for the linear and nonlinear neural network (LNN
and NLNN) models extended to consider ethnicity information.

| | LNN | NLNN |
|---|---|---|
| Ethnic Group | AUC Score (95% C.I.) | AUC Score (95% C.I.) |
| Black/African American | 0.806 (0.799-0.813) | 0.806 (0.800-0.812) |
| Asian | 0.796 (0.790-0.802) | 0.797 (0.791-0.803) |
| Latina | 0.804 (0.798-0.810) | 0.805 (0.799-0.811) |
| Caucasian | 0.799 (0.792-0.806) | 0.801 (0.794-0.809) |

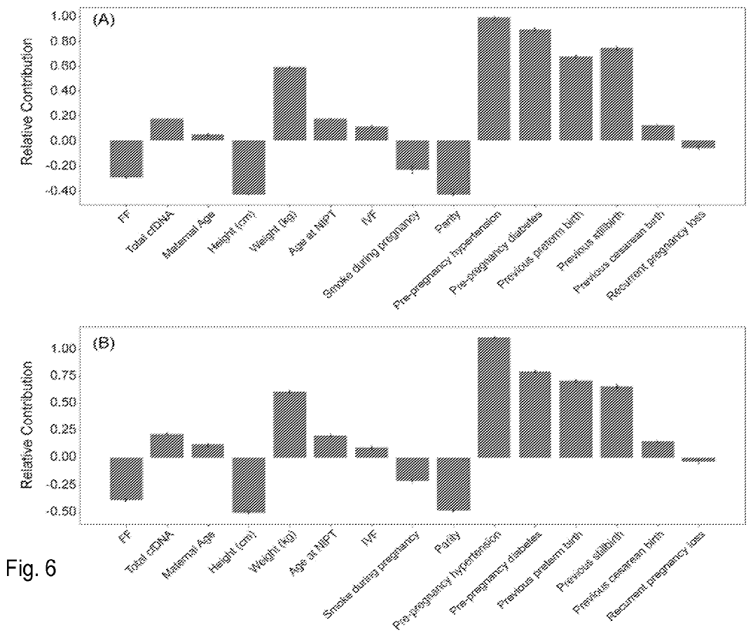

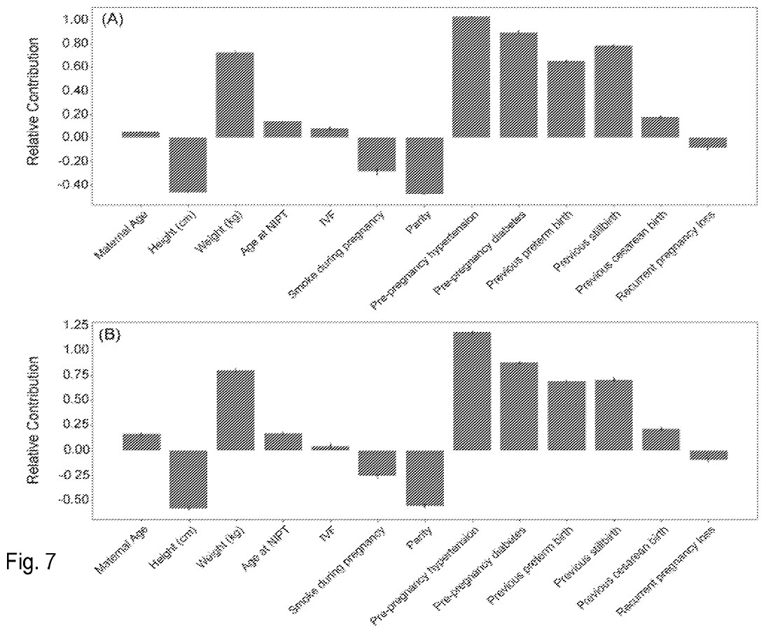

Feature Importance: In example implementations, and with reference to FIGS. 6 and 7, the input features that were the major contributors to both the LNN and NLNN models' predictions were maternal weight, chronic hypertension, pre-pregnancy diabetes, previous preterm birth, and previous stillbirth. Parity and increasing height showed a strong negative correlation with preterm preeclampsia in both models. When the cfDNA features were omitted from the model, the remaining 13 features adapted to compensate for their absence (FIG. 7). Notably, for early onset preeclampsia, a larger relative contribution from the IVF feature (in vitro fertilization) was observed, as well as a smaller relative contribution from the predictive feature related to pre-pregnancy diabetes (BDIABTYP_PRE) after omission of the cfDNA input variables. It is worth mentioning that the use of matrix multiplication (See Methods section) to assess the contribution of the input variables for the NLNN model in the presence of reLu hidden units is justified by the fact that in the example optimized models, all the arguments for the “hidden” activation functions in the optimized models are non-negative. FIG. 7, which shows similar bar-plots built on the results obtained from the reduced models where the cfDNA features were omitted, illustrates how the relative contributions of the remaining thirteen features “adapt” to compensate for the absence of the missing cfDNA features. The same plots for term preeclampsia show similar trends, whereas, for the early onset preeclampsia outcome a larger relative contribution from the IVF feature (in vitro fertilization) was observed, as well as a smaller relative contribution from the predictive feature related to pregestational diabetes (BDIABTYP_PRE).

The example implementations discussed above show, in various implementations, that both linear and nonlinear neural network models using patient characteristics collected at the first antenatal visit and features available from SNP-based cfDNA screening (total cfDNA and FF) can be used to predict preterm preeclampsia with good levels of performance (expected AUC score=0.80, 58% sensitivity for a 15% screen positive rate). The ability of these example models to use only routinely collected clinical data, mostly of a binary nature, has practical and economic advantages for clinical implementations.

In various alternative implementations, mean arterial pressure could significantly increase predictive performance for preterm preeclampsia when added to the model in addition to the other features discussed, or in place of one or more of the discussed features. Blood pressure was not collected as part of SMART but is collected in the first trimester of pregnancy as part of routine clinical practice and could be incorporated in alternative implementations of the disclosed models.

In the disclosed analysis, cfDNA features (total cfDNA and FF) were both associated with risk for preeclampsia. In various embodiments, FF and cfDNA particularly contributed to model performance for early onset preeclampsia. This finding of biomarker performance for earlier onset disease may reflect a different pathogenic process underlying early and later onset preeclampsia.

Advantageously, example implementations of the disclosed approach develop a predictive model based only on easily acquired, routinely collected clinical data and cfDNA data from non-invasive prenatal screening, all of which are part of routine clinical care early in pregnancy in the US. Without the need for other biomarkers (e.g., maternal serum biomarkers) and ultrasound (e.g., uterine artery Doppler), implementations of the disclosed approach have the advantage of maximizing routinely collected input features without adding the expense of biomarkers and ultrasound that are not part of the current clinical pathway for pregnancy care in the US. The disclosed approach could therefore be incorporated into clinical care with minimal resource use and inconvenience for patients.

While the disclosure has been described with respect to specific embodiments, one skilled in the art will recognize that numerous modifications are possible. For instance, although specific examples of rules (including triggering conditions and/or resulting actions) and processes for generating suggested rules are described, other rules and processes can be implemented. Embodiments of the disclosure can be realized using a variety of computer systems and communication technologies including but not limited to specific examples described herein.

Embodiments of the present disclosure can be realized using any combination of dedicated components and/or programmable processors and/or other programmable devices. The various processes described herein can be implemented on the same processor or different processors in any combination. Where components are described as being configured to perform certain operations, such configuration can be accomplished, e.g., by designing electronic circuits to perform the operation, by programming programmable electronic circuits (such as microprocessors) to perform the operation, or any combination thereof. Further, while the embodiments described above may make reference to specific hardware and software components, those skilled in the art will appreciate that different combinations of hardware and/or software components may also be used and that particular operations described as being implemented in hardware might also be implemented in software or vice versa.

Computer programs incorporating various features of the present disclosure may be encoded and stored on various computer readable storage media; suitable media include magnetic disk or tape, optical storage media such as compact disk (CD) or DVD (digital versatile disk), flash memory, and other non-transitory media. Computer readable media encoded with the program code may be packaged with a compatible electronic device, or the program code may be provided separately from electronic devices (e.g., via Internet download or as a separately packaged computer-readable storage medium).

Thus, although the disclosure has been described with respect to specific embodiments, it will be appreciated that the disclosure is intended to cover all modifications and equivalents within the scope of the following claims.

As used herein, “routine” prenatal visits to a healthcare provider are “checkup” type visits to a healthcare provider for the purpose of maintaining health and wellness. Routine visits are intended to prevent adverse health outcomes rather than for treatment of existing symptoms or illnesses. Routine prenatal visits are recommended for populations of pregnant individuals in general, as opposed to being prompted by patient-specific health concerns.

The following include potential embodiments of the disclosed approach, representative of other examples that are not intended to be limiting in any way:

Embodiment A1: A method of applying, by a computing system, a predictive machine learning classifier to generate a prediction of a patient developing a preeclampsia condition during a current pregnancy, the predictive machine learning classifier comprising one or more artificial neural networks, wherein generating the prediction comprises: obtaining patient data by at least one of (A) performing a non-invasive prenatal screening (NIPS) on the patient, or (B) receiving, by the computing system through a communications network, data from a health record system, wherein the patient data comprises (i) a set of health characteristics, and (ii) one or more cell-free DNA (cfDNA) metrics; feeding the patient data to the predictive machine learning classifier to obtain a prediction of the patient developing the preeclampsia condition during the current pregnancy; and providing, by the computing system, the prediction for care of the patient, wherein providing the prediction comprises at least one of transmitting, by the computing system through the communications network, the prediction to at least one of the health record system or a computing device associated with a healthcare provider, wherein the predictive machine learning classifier was trained by applying one or more deep learning techniques to data on subjects in a cohort of pregnant subjects, the data comprising, for each pregnant subject: (A) a frst feature set indicative of one or more outcomes of one or more prior pregnancies of the pregnant subject; (B) a second feature set comprising one or more health indicators for the pregnant subject; and (C) a third feature set based on one or more cfDNA measurements for the pregnant subject.

Embodiment A2: The method of Embodiment A1, wherein all of the patient data used to obtain the prediction is collected non-invasively during one or more routine medical visits of the patient.

Embodiment A3: The method of either Embodiment A1 or A3, wherein the patient data used to obtain the prediction does not include ultrasound data.

Embodiment A4: The method of any of Embodiments A1-A3, wherein the preeclampsia condition is early onset preterm preeclampsia.

Embodiment A5: The method of any of Embodiments A1-A4, further comprising generating, by the computing system, a training dataset based on the first, second, and third feature sets.

Embodiment A6: The method of any of Embodiments A1-A5, further comprising training, by the computing system, the predictive machine learning classifier using the data on the subjects in the cohort of pregnant subjects.

Embodiment A7: The method of any of Embodiments A1-A6, wherein the predictive machine learning classifier comprises one or more rectified linear units (reLu).

Embodiment A8: The method of any of Embodiments A1-A7, wherein the predictive machine learning classifier comprises a hidden layer with a same dimension as an input layer.

Embodiment A9: The method of any of Embodiments A1-A8, wherein the one or more deep learning techniques comprises using one or more regularizers.

Embodiment B1: A method comprising generating a predictive machine learning classifier for a preeclampsia condition in pregnant patients, the predictive machine learning classifier comprising one or more artificial neural networks, wherein generating the predictive machine learning classifier comprises: generating, by a computing system, a training dataset using data on subjects in a cohort of pregnant subjects, the training dataset comprising, for each pregnant subject: (A) a first feature set indicative of one or more outcomes of one or more prior pregnancies of the subject; (B) a second feature set corresponding to one or more health indicators for the subject; and (C) a third feature set corresponding to one or more cfDNA metrics; applying, by the computing system, one or more deep learning techniques to the training dataset to generate the predictive machine learning classifier, wherein the predictive machine learning classifier is configured to receive, as inputs, patient health characteristics and one or more cfDNA metrics, and output predictions on dcvcloping the precclampsia condition; and providing, by the computing system, the predictive machine learning classifier to generate patient-specific indicators for individual patients, wherein providing the predictive machine learning classifier comprises at least one of (i) transmitting, by the computing system through a communications network, the predictive machine learning classifier to a second computing system, or (ii) storing, in a non-transient computer-readable storage medium, the predictive machine learning classifier accessible for subsequent use by a healthcare provider.

Embodiment B2: The method of Embodiment B1, wherein the predictive machine learning classifier comprises one or more hidden rectified linear units (reLu).

Embodiment B3: The method of either Embodiment B1 or B2, wherein the predictive machine learning classifier comprises a hidden layer with a same dimension as an input layer.

Embodiment B4: The method of any of Embodiments B1-B3, wherein applying the one or more deep learning techniques comprises using one or more regularizers.

Embodiment B5: The method of any of Embodiments B1-B4, wherein the one or more regularizers comprises an L2 regularizer.

Embodiment B6: The method of any of Embodiments B1-B5, further comprising using the predictive machine learning classifier to evaluate a patient during a current pregnancy by: performing a non-invasive prenatal screening (NIPS) on the patient; feeding data to the predictive machine learning classifier to obtain a prediction for development of the preeclampsia condition during the current pregnancy, the data comprising data from the NIPS; and using the prediction in delivery of healthcare to the patient, wherein all of the data provided to the predictive machine learning classifier to obtain the prediction is collected non-invasively during one or more routine medical visits of the patient.

Embodiment B7: The method of any of Embodiments B1-B6, further comprising using the predictive machine learning classifier to evaluate a patient during a current pregnancy by: receiving, from a health record system, data corresponding to one or more prior pregnancies of the patient, one or more health characteristics, and one or more cfDNA metrics; feeding the data to the predictive machine learning classifier to obtain a prediction for development of the preeclampsia condition during the current pregnancy; and using the prediction in delivery of healthcare to the patient, wherein all the data input into the predictive machine learning classifier to obtain the prediction is collected non-invasively during one or more routine medical visits of the patient.

Embodiment B8: The method of any of Embodiments B1-B7, wherein the preeclampsia condition is an early onset preterm preeclampsia.

Embodiment C1: A computing system configured to apply a predictive machine learning classifier to generate a prediction of a patient developing a preeclampsia condition during a current pregnancy, the predictive machine learning classifier comprising one or more artificial neural networks, the computing system comprising one or more processors configured to: receive, during the current pregnancy of the patient, through a communications network, patient data comprising a set of health characteristics and one or more cfDNA measurements; feed the patient data to the predictive machine learning classifier to obtain a prediction of the patient developing the preeclampsia condition during the current pregnancy; and provide the prediction for care of the patient, wherein providing the prediction comprises transmitting, through the communications network, the prediction to at least one of a health record system or a computing device associated with a healthcare provider, wherein the predictive machine learning classifier was trained by applying one or more deep learning techniques to data on subjects in a cohort of pregnant subjects, the training data comprising, for each pregnant subject: (A) a first feature set indicative of one or more outcomes of one or more prior pregnancies of the pregnant subject; (B) a second feature set corresponding to one or more health indicators for the pregnant subject; and (C) a third feature set corresponding to one or more cell-free DNA metrics for the pregnant subject.

Embodiment C2: The computing system of Embodiment C1, wherein all of the patient data used to obtain the prediction is collected non-invasively during one or more routine medical visits of the patient.

Embodiment C3: The computing system of either Embodiment C1 or C2, wherein the preeclampsia condition is early onset preterm preeclampsia.

Embodiment C4: The computing system of any of Embodiments C1-C3, wherein the one or more processors are further configured to generate the training dataset and apply the deep learning techniques to the training dataset to generate the predictive machine learning classifier.

Embodiment C5: The computing system of any of Embodiments C1-C4, wherein the one or more processors are further configured to receive at least a subset of the health data from the health record system via the communications network.

Embodiment C6: The computing system of any of Embodiments C1-C5, wherein the one or more processors are further configured to receive at least a subset of the health data via a software application running on the computing device associated with the healthcare provider.

Embodiment D1: A method for developing machine learning models useful for predicting development of preterm preeclampsia, comprising: identifying, by one or more processors, for each subject of a plurality of subjects, data comprising at least three of maternal age, maternal weight, gestational age, total cell free DNA, and a fetal fraction percentile relative to a fetal fraction distribution corresponding to the maternal weight and gestation age for the sample, the data further comprising one of a first label indicating that the subject developed preterm preeclampsia or a second label indicating that the subject did not develop preterm preeclampsia; and training, by the one or more processors, using the identified data, a machine learning model to generate values indicative of a risk that subjects develop preterm preeclampsia.

Embodiment D2: The method of Embodiment D1, wherein the machine-learning model includes at least one of a regularized logistic regression model or a feedforward neural network.

Embodiment D3: The method of either Embodiment D1 or D2, wherein the model includes a binary classifier.

Embodiment D4. The method of any of Embodiments D1-D3, wherein the model is trained and validated using a stratified, repeated k-fold cross validation (SRKFCV) approach.

Embodiment D5. The method of any of Embodiments D1-D4, wherein the machine-learning model is trained by: a) initializing a model parameter accumulator and a score accumulator; b) assigning a first portion of a data set to a training data set and a second portion of the data set to a validation data set; c) oversampling the minority class in the training data set to generate an oversampled training data set d) oversampling the minority class in the validation data set to generate an oversampled validation data set; e) fitting the machine-learning model on the oversampled training data set; f) generating a predicted outcome on the oversampled validation data set; g) determining an accuracy score and an area under a curve (AUC) score based on the predicted outcome; and h) storing, in one or more data structures, one or more model parameters in the model parameter accumulator and the accuracy score and the AUC score in the score accumulator.

Embodiment D6. The method of Embodiment D5, wherein steps b)-h) are repeated a predetermined number of times.

Embodiment D7. The method of Embodiment D5, wherein the machine-learning model is a logistic regression and a regularization constant is a hyperparameter.

Embodiment D8. The method of Embodiment D5, wherein the machine-learning model is a feedforward neural network and a regularization constant, an activation function, a number and size of hidden layers, and a cost function are optimized.

Embodiment E1. A method for predicting development of preterm preeclampsia, comprising: identifying, by one or more processors, for a subject, input data comprising at least three of maternal age, maternal weight, gestational age, total cell free DNA, and a fetal fraction percentile relative to a fetal fraction distribution corresponding to the maternal weight and gestation age for the sample; determining, by the one or more processors, responsive to inputting the input data in a machine-learning model trained using data to generate values indicative of a risk that subjects develop preterm preeclampsia, a value indicative of a risk of the subject developing preterm preeclampsia; and providing, by the one or more processors, for presentation, an output based on the value.

Embodiment E2: The method of Embodiment E1, wherein the machine-learning model includes at least one of a regularized logistic regression model or a feedforward neural network.

Embodiment E3: The method of either Embodiment E1 or E2, wherein the model includes a binary classifier.

Embodiment E4: The method of any of Embodiments E1-E3, wherein the model is trained and validated using a stratified, repeated k-fold cross validation (SRKFCV) approach.

Embodiment E5: The method of any of Embodiments E1-E4, wherein the machine-learning model is trained by: a) initializing a model parameter accumulator and a score accumulator, b) assigning a first portion of a data set to a training data set and a second portion of the data set to a validation data set; c) oversampling the minority class in the training data set to generate an oversampled training data set; d) oversampling the minority class in the validation data set to generate an oversampled validation data set; e) fitting the machine-learning model on the oversampled training data set; f) generating a predicted outcome on the oversampled validation data set; g) determining an accuracy score and an area under a curve (AUC) score based on the predicted outcome; and h) storing, in one or more data structures, one or more model parameters in the model parameter accumulator and the accuracy score and the AUC score in the score accumulator.

Embodiment E6: The method of Embodiment E5, wherein steps b)-h) are repeated a predetermined number of times.

Embodiment E7: The method of Embodiment E5, wherein the machine-learning model is a logistic regression and a regularization constant is a hyperparameter.

Embodiment E8: The method of Embodiment E5, wherein the machine-learning model is a feedforward neural network and a regularization constant, an activation function, a number and size of hidden layers, and a cost function are optimized.

Embodiment F: A computing system comprising one or more processors configured to implement any of Embodiments A1 to B8 or D1 to E8.

Embodiment E: A non-transitory computer-readable storage medium with instructions configured to cause one or more processors of a computing system to implement any of Embodiments A1 to B8 or D1 to E8.

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

1. A method of applying, by a computing system, a predictive machine learning classifier to generate a prediction of a patient developing a preterm preeclampsia during a current pregnancy, the predictive machine learning classifier comprising one or more artificial neural networks, wherein generating the prediction comprises:
obtaining patient data by at least one of (A) performing a non-invasive prenatal screening (NIPS) on the patient, or (B) receiving, by the computing system through a communications network, data from a health record system, wherein the patient data comprises (i) a set of health characteristics, and (ii) one or more cell-free DNA (cfDNA) metrics;
feeding the patient data to the predictive machine learning classifier to obtain a prediction of the patient developing the preterm preeclampsia during the current pregnancy; and
providing, by the computing system, the prediction for care of the patient, wherein providing the prediction comprises at least one of transmitting, by the computing system through the communications network, the prediction to at least one of the health record system or a computing device associated with a healthcare provider,
wherein the predictive machine learning classifier was trained by applying one or more deep learning techniques to data on subjects in a cohort of pregnant subjects, the data comprising, for each pregnant subject: (A) a first feature set indicative of one or more outcomes of one or more prior pregnancies of the pregnant subject; (B) a second feature set comprising one or more health indicators for the pregnant subject; and (C) a third feature set based on one or more cfDNA measurements for the pregnant subject.

2. The method of claim 1, wherein all of the patient data used to obtain the prediction (i) is collected non-invasively during one or more routine medical visits of the patient, and (ii) does not include ultrasound data.

3. The method of claim 1, wherein the preterm preeclampsia is an early onset preterm preeclampsia.

4. The method of claim 1, further comprising generating, by the computing system, a training dataset based on the first, second, and third feature sets.

5. The method of claim 1, further comprising training, by the computing system, the predictive machine learning classifier using the data on the subjects in the cohort of pregnant subjects.

6. The method of claim 1, wherein the predictive machine learning classifier comprises (i) one or more rectified linear units (reLu), and (ii) a hidden layer with a same dimension as an input layer, wherein the one or more deep learning techniques comprises using one or more regularizers.

7. A method comprising generating a predictive machine learning classifier for preterm preeclampsia in pregnant patients, the predictive machine learning classifier comprising one or more artificial neural networks, wherein generating the predictive machine learning classifier comprises:
generating, by a computing system, a training dataset using data on subjects in a cohort of pregnant subjects, the training dataset comprising, for each pregnant subject:
(A) a first feature set indicative of one or more outcomes of one or more prior pregnancies of the subject;
(B) a second feature set corresponding to one or more health indicators for the subject; and
(C) a third feature set corresponding to one or more cfDNA metrics;
applying, by the computing system, one or more deep learning techniques to the training dataset to generate the predictive machine learning classifier, wherein the predictive machine learning classifier is configured to receive, as inputs, patient health characteristics and one or more cfDNA metrics, and output predictions on developing preterm preeclampsia; and
providing, by the computing system, the predictive machine learning classifier to generate patient-specific indicators for individual patients, wherein providing the predictive machine learning classifier comprises at least one of (i) transmitting, by the computing system through a communications network, the predictive machine learning classifier to a second computing system, or (ii) storing, in a non-transient computer-readable storage medium, the predictive machine learning classifier accessible for subsequent use by a healthcare provider.

8. The method of claim 7, wherein the predictive machine learning classifier comprises one or more hidden rectified linear units (reLu).

9. The method of claim 7, wherein the predictive machine learning classifier comprises a hidden layer with a same dimension as an input layer.

10. The method of claim 7, wherein applying the one or more deep learning techniques comprises using one or more regularizers.

11. The method of claim 10, wherein the one or more regularizers comprises an L2 regularizer.

12. The method of claim 7, further comprising using the predictive machine learning classifier to evaluate a patient during a current pregnancy by:
performing a non-invasive prenatal screening (NIPS) on the patient;
feeding data to the predictive machine learning classifier to obtain a prediction for development of the preterm preeclampsia during the current pregnancy, the data comprising data from the NIPS; and
using the prediction in delivery of healthcare to the patient, wherein all of the data provided to the predictive machine learning classifier to obtain the prediction is collected non-invasively during one or more routine medical visits of the patient.

13. The method of claim 7, further comprising using the predictive machine learning classifier to evaluate a patient during a current pregnancy by:
receiving, from a health record system, data corresponding to one or more prior pregnancies of the patient, one or more health characteristics, and one or more cfDNA metrics;
feeding the data to the predictive machine learning classifier to obtain a prediction for development of the preterm preeclampsia during the current pregnancy; and
using the prediction in delivery of healthcare to the patient, wherein all the data input into the predictive machine learning classifier to obtain the prediction is collected non-invasively during one or more routine medical visits of the patient.

14. The method of claim 1, wherein the preterm preeclampsia is an early onset preterm preeclampsia.

15. A computing system configured to apply a predictive machine learning classifier to generate a prediction of a patient developing preterm preeclampsia during a current pregnancy, the predictive machine learning classifier comprising one or more artificial neural networks, the computing system comprising one or more processors configured to:
receive, during the current pregnancy of the patient, through a communications network, patient data comprising a set of health characteristics and one or more cfDNA measurements;
feed the patient data to the predictive machine learning classifier to obtain a prediction of the patient developing the preterm preeclampsia during the current pregnancy; and
provide the prediction for care of the patient, wherein providing the prediction comprises transmitting, through the communications network, the prediction to at least one of a health record system or a computing device associated with a healthcare provider;
wherein the predictive machine learning classifier was trained by applying one or more deep learning techniques to data on subjects in a cohort of pregnant subjects, the training data comprising, for each pregnant subject: (A) a first feature set indicative of one or more outcomes of one or more prior pregnancies of the pregnant subject; (B) a second feature set corresponding to one or more health indicators for the pregnant subject; and (C) a third feature set corresponding to one or more cell-free DNA metrics for the pregnant subject.

16. The computing system of claim 15, wherein all of the patient data used to obtain the prediction is collected non-invasively during one or more routine medical visits of the patient.

17. The computing system of claim 15, wherein the preterm preeclampsia is an early onset preterm preeclampsia.

18. The computing system of claim 15, wherein the one or more processors are further configured to generate the training dataset and apply the deep learning techniques to the training dataset to generate the predictive machine learning classifier.

19. The computing system of claim 15, wherein the one or more processors are further configured to receive at least a subset of the health data from the health record system via the communications network.

20. The computing system of claim 15, wherein the one or more processors are further configured to receive at least a subset of the health data via a software application running on the computing device associated with the healthcare provider.
